(19) United States
(12) Patent Application Publication
(10) Pub. No.: US 2013/0338072 A1
(43) Pub. Date: Dec. 19, 2013

(54) METHODS FOR TREATING ORGANS

(71) Applicants: Leo RUBIN, Suffern, NY (US); Shaker MOUSA, Wynantskill, NY (US)

(72) Inventors: Leo RUBIN, Suffern, NY (US); Shaker MOUSA, Wynantskill, NY (US)

(21) Appl. No.: 13/918,666

(22) Filed: Jun. 14, 2013

Related U.S. Application Data
(60) Provisional application No. 61/660,657, filed on Jun. 15, 2012.
(60) Provisional application No. 61/660,705, filed on Jun. 16, 2012.

(51) Int. Cl. A61K 38/22; A61K 31/198

(52) U.S. Cl. CPC A61K 38/2278; A61K 31/198
USPC 514/13.1

(57) ABSTRACT

The present invention relates to methods of preventing or reducing the risk of acute and chronic rejection of transplanted organs. The invention relates to the administration of T3, VIP, and T3/VIP nanoparticles to a donor organ in order to prevent or reduce the risk of rejection, e.g. acute or chronic rejection.

### RELATED APPLICATIONS

The application claims the benefit of U.S. Provisional Application 61/660,657 filed Jun. 15, 2012, and U.S. Provisional Application 61/660,705, filed Jun. 16, 2012, the contents of each of which are incorporated herein by reference.

### TECHNICAL FIELD

The present invention relates to methods of preventing or reducing the risk of acute and chronic rejection of transplanted organs. Specifically, the invention relates to the administration of T3, VIP, and T3/VIP nanoparticles to a donor organ in order to prevent or reduce the risk of rejection, e.g. acute or chronic rejection.

### BACKGROUND OF THE INVENTION

The immunological response directed against the transplanted foreign tissue, such as a donor organ, must be controlled if the tissue is to survive and function. It is known that the normally functioning immune system of the transplant recipient recognizes the transplanted organ as “non-self” tissue and thereafter mounts an immune response to the presence of the transplanted organ or donor organ. Left unchecked, the immune response will ultimately result in loss of biological functioning or death of the transplanted organ. Transplant rejection remains the leading impediment to long term graft survival in humans. Current immunosuppressive therapy used to treat rejection reactions are directed toward the suppression of T and B cell activity.

Tissue and organ transplant recipients are generally treated with one or more cytotoxic agents in an effort to suppress the transplant recipient's immune-response against the transplanted organ or tissue. For example, cyclosporin A (e.g., Neoral® or Sandimmune®), a cyclic peptide consisting of 11 amino acid residues is currently used to administer to the recipients of kidney, liver, pancreas and heart allografts (i.e., wherein donor and recipient are of the same species). However, administration of cyclosporin A is not without drawbacks as the drug can cause kidney and liver toxicity as well as hypertension. Moreover, the use of cyclosporin A can lead to malignancies (such as lymphoma) and lead to opportunistic infection due to the systemic immunosuppression it induces in patients receiving long, term treatment with the drug, i.e., the normal protective immune response of the host to pathogenic microorganisms is down-regulated thereby increasing the risk of infections caused by such microorganisms.

Another significant issue with organ transplantation is acute and chronic rejection. Unfortunately, currently available immunosuppressive agents such as cyclosporin A fail to prevent either acute or chronic refractory rejection. Chronic rejection poses formidable hurdles for existing immunosuppressant therapies. For example, many cadaver renal transplants do not function at ten years post-transplant. Transplant vasculopathy, induced by chronic rejection and ischemia, may be a cause of cardiac transplant graft loss after the first year post transplant. Moreover, current post-transplantation therapy requires continuous (e.g. daily) administration of an anti-rejection agent for the duration of the transplant recipient's life.

Acute rejection may occur to some degree in most transplants. However, failure to attenuate acute rejection, or recurrent rejection episodes, may be risk factors for the development in some forms of chronic rejection. Ulf P. Neumann, Jan M. Langrehr and Peter Neuhaus “Chronic Rejection after Human Liver Transplantation”, Graft (2002); 5; 102

Historically, chronic vascular rejection has been described as repetitive endothelial injury leading to intimal proliferation, hypertrophy and subsequent luminal occlusion (Tilney et al. (1995) Transplantation. Proc. 27:2123-2125; Tilney et al. (1991) Transplantation Proc. 52:389-398). Some researchers have proposed inflammatory, humoral, cellular, and cytokine-related non-specific scarring mechanisms as etiologies of chronic rejection (Hayry et al. (1996) Transplantation Proc. 28:2337-2338; Tilney et al. (1995) Transplantation Proc. 27:2123-2125; Tilney et al. (1991) Transplantation Proc. 52:389-398

These isograft losses are believed to be a consequence of injury during preservation and reperfusion. Injury from multiple etiologies activates thrombotic and inflammatory cascades in the vascular wall that converge, initiating a rapid pervasive response which stimulates cellular migration, invasion and proliferation at sites of vessel injury (Aziz et al. (1995) Lung Transplant 14:S123-S136; Libby et al., (1992) Circulation 86:Supp:III:47-52).

Triiodothyronine, also known as T3, is a thyroid hormone. Thyroid-stimulating hormone (TSH) activates the production of thyroxine (T4) and T3. T4 is converted to T3 by deiodination. T3 affects a variety of body processes, including body temperature, growth, and heart rate. T3 has important effects on cardiac tissue. Thyroid hormones, notably T3, modulate ventricular function via genomic and non-genomic mechanisms. Cardiac stress events (cardiac arrest, myocardial infarction, etc.) are associated with steep reductions in serum T3 levels. Post resuscitation T3 level correlates highly with survival rate. T3 additionally has cardiostimulatory properties: it increases the cardiac output by increasing the heart rate and force of contraction. Overall, there is reason to believe that early bolus T3 injection could increase chances of resuscitating cardiac arrest victims, and that elevating T3 serum concentration could increase prospects of survival to hospital discharge.

T3 is not currently approved for this indication, however, and current formulations of T3 are not well suited for this purpose. Triostat® requires refrigeration, making it somewhat impractical for emergency use. Also, the concentration is low for what is needed to treat cardiac arrest. T3-albumin formulation have been described but are difficult to make, and like Triostat®, have poor stability and are poorly suited for quick administration in an emergency setting.

Vasoactive intestinal peptide (VIP) is a peptide hormone containing 28 amino acid residues, produced in many areas of the human body including the gut, pancreas and suprachiasmatic nuclei of the hypothalamus in the brain. In humans, the vasoactive intestinal peptide is encoded by the VIP gene. Various synthetic forms of VIP or VIP from other mammalian sources are known. VIP causes vasodilatation, lowers arterial blood pressure, stimulates myocardial contractility, increases glycogenolysis and relaxes the smooth muscle of trachea, stomach and gall bladder. VIP is a potent dilator of the pulmonary and coronary arteries, and has great potential to reduce pulmonary arterial hypertension and at the same time enhance cardiac function. VIP is also known to dilate the cardiac arteries and to enhance cardiac function. VIP is therefore useful to treat acute myocardial infarction and to treat heart failure resulting from myocardial infarction. To date, however, it has not been used as a therapeutic because it has a half-life (T_{1/2}) in the blood of less than two minutes.

There is currently an unmet need for effective methods and/or compounds that can reduce the risk of rejection in a transplanted organ.

### SUMMARY OF THE INVENTION

The present invention provides a method of administering an effective amount of T3 nanoparticles (e.g., T3 nanoparticle formulation) and/or VIP nanoparticles (e.g., VIP nanoparticle formulation) in order to treat transplant organs or organs intended for transplantation. It is contemplated that the organs are donor organs, e.g. heart, lung, combination heart/lung, kidney, liver. In one embodiment the donor organs are human.

In another embodiment the donor organs are non-human.

In one embodiment an effective amount of T3 nanoparticles and/or VIP nanoparticles can be administered to an organ prior to transplantation.

It is also contemplated that an effective amount of T3 nanoparticles and/or VIP nanoparticles can be administered to an organ contemporaneously with transplantation.

In one embodiment the T3 nanoparticles and/or VIP nanoparticles may be administered to an organ both contemporaneously and prior to transplantation.

In one embodiment the T3 nanoparticles and/or VIP nanoparticles may be administered to the recipient of a donor organ following transplant.

Without being bound by theory, one reason for the effectiveness of the administration of T3 nanoparticles and/or VIP nanoparticles, may be that the compounds can have potent vasodilatory action which may allow donor organs to avoid hypoxic and/or ischemic injury prior to or during transplantation, which may occur prior to or subsequent to reperfusion. This, in turn, may avoid reperfusion injury (e.g., either acute and/or chronic reperfusion injury) and also decrease the risk that that organ will be rejected (e.g., either acute or chronic organ rejection). Administration of either T3 and/or VIP has typically been difficult due to the compounds respective issues regarding stability and short-half life. However, by utilizing the nanoparticles discussed herein, VIP and T3 may now be used effectively to protect donor organs as the nanoparticles function to increase and improve stability and half-life of both T3 and VIP respectively. Without being bound to any theory, it is believed that the administration of T3 and/or VIP nanoparticles enhances perfusion and reduces the risk of rejection of a donor organ, e.g. acute and/or chronic rejection.

In one embodiment of the invention the composition comprising a T3 and/or VIP nanoparticles are administered alone. In another embodiment the T3 and/or VIP nanoparticles are administered with an anti-rejection agent.

In one embodiment the administration of the T3 nanoparticles and/or a VIP nanoparticles reduces the risk, incidence and/or extent of ischemic/reperfusion injury in a donor or transplant organ. It is contemplated that the reduction of ischemic/reperfusion injury relates to both acute and chronic reperfusion injury. In one embodiment a T3/VIP combination nanoparticle is administered to a donor or transplant organ in order to reduce the risk or rejection (e.g., acute or chronic rejection).

In one embodiment the invention is directed to treating mammalian kidney transplant rejection. Another embodiment of the invention is directed to treating mammalian heart transplant rejection. Still another embodiment of the invention is directed to treating graft rejection of an organ transplanted from one mammalian species to another, distinct mammalian species. In one embodiment of the invention, T3 and/or VIP nanoparticles are delivered in combination with an anti-rejection agent e.g., Cyclosporin A in a manner consistent with conventional methodologies associated with transplantation of mammalian organs in order to treat graft rejection.

### DETAILED DESCRIPTION

It is contemplated that the methods of the present invention include administration of T3 nanoparticles and/or a VIP nanoparticles to human donor organs. In another embodiment, the administration of T3 nanoparticles and/or VIP nanoparticles may be administered to appropriate non-human donor organs. In another embodiment the donor organs are taken from a cadaver.

“Transplant organ” as used herein may refer to an organ that is also a donor organ and/or an organ that is intended for transplantation. “Transplant organ” may refer to a donor organ that has yet to be transferred to a recipient or an organ that has already been transferred from a donor to a recipient. “Donor organ” as used herein may refer to an organ that will be transplanted into a recipient, or already has been transplanted into a recipient, from a donee. The donor organ may refer to an organ taken from a cadaver.

A “corneal transplant” refers to the insertion of a cornea into the eye of a mammal, where the cornea being inserted is not the natural cornea of the mammal. The cornea being inserted may be from a cadaver.

The term “anti-rejection agent” as used herein means any commercially available immunosuppressive pharmaceutical agent which reduces the tendency of a transplanted organ to be rejected by the transplant recipient. Transplant rejection treatment is assessed in accordance with the present invention by one or more of the following organ-dependent parameters decreased coronary graft intimal hyperplasia compared to control grafted vessels; renal function as measured by serial serum creatinine levels; graft survival prolongation; hyalinization and cortical scarring in renal grafts.

“Acute ischemic injury”, “Acute reperfusion injury”, or like terms, refer to a sudden interruption in the blood supply to a tissue, organ, or extremity that, if untreated, can lead to tissue death.

“Chronic ischemic injury”, “Chronic reperfusion injury”, or like terms, refer to, e.g., persistent restriction of blood supply to a tissue that can impair tissue function and result in tissue and organ damage.

“Reduction of the risk of organ rejection”, or like terms, may refer in certain cases to limiting or reducing the likelihood that a donor organ will be rejected. In some cases this phrase may refer to reducing the duration of a rejection response (e.g., acute response). In certain cases this reduction in the rejection duration may result in prolonging the viability and life of an organ or tissue as compared to an untreated organ or tissue. In some cases it is contemplated that the administration of the nanoparticle formulation will reduce the amount of damage to the organ or tissue—relative to an untreated organ or tissue—and consequently reduce the risk that any subsequent organ rejection (e.g., acute or chronic) would render the organ or tissue non-viable.

In one aspect, it is contemplated that the administration of the nanoparticle formulation is sufficient to attenuate a rejection response (e.g., acute or chronic) therefore resulting the increased viability of the donor tissue or organ. In one aspect it is contemplated that the nanoparticle formulation disclosed herein is administered in an amount effective to limit or attenuate any reperfusion injury which, in turn, limits or attenuates any subsequent rejection response (e.g., acute or chronic) relative to an organ that is not treated with a nanoparticle formulation as disclosed herein.

“Reduce the incidence of reperfusion injury”, e.g., acute or chronic, may refer in certain cases to reducing the likelihood or probability that an organ will suffer a reperfusion injury. The reduction is relative to a donor organ which has not been treated.

Anti-rejection agents in accordance with the present invention are contemplated to include immunosuppressive agents. Anti-rejection agents contemplated by the present invention specifically include but are not limited to cyclosporin (e.g., Cyclosporin A, Sandimmune®, Neoral®, (Novartis), Rapimmune® (American Home Products) FK501 (Fujisawa), CELLCEPT® (Roche, Syntex), IMUREK®, SPANIDIN® and PROGRAF®).

The present invention provides for Method I, wherein Method I is a method for preventing or reducing the risk of organ transplant rejection (e.g, acute or chronic rejection) of a donor organ comprising administering or treating a donor organ with an effective amount of a formulation comprising T3, VIP, T3/VIP nanoparticles.
1.1. Method I, wherein the T3, VIP, T3/VIP nanoparticles are administered prior to transplantation of the donor organ.
1.2. Method I, wherein the T3, VIP, T3/VIP nanoparticles are administered contemporaneously with the transplantation of the donor organ.
1.3. Method I, wherein the T3, VIP, T3/VIP nanoparticles are administered both prior to, and contemporaneously, with the transplantation of the donor organ.
1.4. Method I or any of methods 1.1-1.3, wherein an effective amount of a T3 nanoparticle formulation is administered.
1.5. Method I or any of methods 1.1-1.4, wherein an effective amount of VIP nanoparticle formulation is administered.
1.6. Method I or any of methods 1.1-1.5, wherein an effective amount of a combination T3/VIP nanoparticle formulation is administered.
1.7. Method I or any of the forgoing methods wherein an effective amount of T3 and/or VIP nanoparticles are administered in combination with an additional thyroid hormone.
1.8. Method of method 1.8 wherein the additional thyroid hormone is thyroxine (T4).
1.9. Method I or any of the preceding methods wherein the donor organ is selected from the group consisting of: kidney, heart, lung, combined heart/lung, liver, pancreas, islet cell, face, hand, uterus, bone marrow, eye, cornea, trachea, bowel, e.g. small bowel, skin, muscles or limb, oesophagus, and nervous tissue.
1.10. Method I, or any of the preceding methods wherein the administration T3 and/or VIP nanoparticles reduce the incidence of acute reperfusion injury by about 25%.
1.11. Method I, or any of the preceding methods wherein the administration T3 and/or VIP nanoparticles reduce the incidence of acute reperfusion injury by about 50%.
1.12. Method I, or any of the preceding methods wherein the administration T3 and/or VIP nanoparticles reduce the incidence of acute reperfusion injury by about 75%.
1.13. Method I, or any of the preceding methods wherein the administration T3 and/or VIP nanoparticles reduce the incidence of acute reperfusion injury by about 100%.
1.14. Method I, or any of the preceding methods wherein the administration of T3 and/or VIP nanoparticles reduce the incidence of chronic reperfusion injury by about 25%.
1.15. Method I, or any of the preceding methods wherein the administration of T3 and/or VIP nanoparticles reduce the incidence of chronic reperfusion injury by about 50%.
1.16. Method I, or any of the preceding methods wherein the administration of T3 and/or VIP nanoparticles reduce the incidence of chronic reperfusion injury by about 75%.
1.17. Method I, or any of the preceding methods wherein the administration of T3 and/or VIP nanoparticles reduce the incidence of chronic reperfusion injury by about 100%.
1.18. Method I, or any of the preceding methods, wherein the T3 and/or VIP nanoparticles are administered in combination with an anti-rejection agent.
1.19. Method of Method 1.18, wherein said anti-rejection agent is selected from the group consisting of: to cyclosporin (e.g., Cyclosporin A, Sandimmune®, Neoral®, (Novartis), Rapimmune® (American Home Products) FK501 (Fujisawa), and CELLCEPT® (Roche, Syntex), IMUREK®, SPANIDIN® and PROGRAF®).
1.20. Method I or any of the preceding methods, wherein the T3 nanoparticles and/or VIP nanoparticles are administered in a balanced isotonic solution.
1.21. Method of Method 1.23, wherein the balanced isotonic solution comprises sodium, potassium, calcium, magnesium, and bicarbonate ions.
1.22. Method I or any of the preceding methods wherein the administration of T3 nanoparticles and/or a VIP nanoparticles comprises perfusion of a donor organ.
1.23. Method I or any of the preceding methods wherein the administration of T3 nanoparticles and/or a VIP nanoparticles comprises a first perfusion and then a subsequent re-perfusion.
1.24. Method I or any of the preceding methods wherein the administration of T3 nanoparticles and/or VIP nanoparticles comprises a continuous perfusion or infusion.
1.25. Method I or any of the preceding methods wherein the administration of T3 nanoparticles and/or VIP nanoparticles comprises bathing the donor organ in a solution or formulation comprising T3 and/or VIP nanoparticles, prior to transplantation.
1.26. Method I or any of the preceding methods wherein the amount of T3 nanoparticles administered is between about 0.5 μg/kg-1 mg/kg.
1.27. Method I or any of the preceding methods wherein the T3 nanoparticles are administered as a bolus in an amount from about 1 μg-1 mg.
1.28. Method of method 1.30 wherein the T3 nanoparticles are administered as a bolus from about 1 μg-100 μg. (1, 5, 10, 20, 30, 40, 50, 60, 70, 80, 90, and 100 μg/hour)
1.29. Method of method 1.27 or 1.28 wherein the T3 nanoparticles are administered as a bolus is about 1 μg-10 μg. (e.g., 1, 2, 3, 4, 5, 6, 7, 8, 9, or 10 μg/hour)
1.30. Method I or any of the preceding methods wherein the T3 nanoparticles are administered as an infusion in an amount from about 1 μg/hour-1 mg/hour.
1.31. Method of method 1.30 wherein the T3 nanoparticles are administered as an infusion from about 1 μg/hour-100 μg/hour. (1, 5, 10, 20, 30, 40, 50, 60, 70, 80, 90, and 100 μg/hour)
1.32. Method of method 1.30 or 1.31 wherein the T3 nanoparticles are administered as an infusion is from about 1 μg/hour-10 μg/hour. (e.g., 1, 2, 3, 4, 5, 6, 7, 8, 9, or 10 μg/hour)
1.33. Method I or any of the preceding methods, wherein the donor organ is the from the same species as the recipient.
1.34. Method I or any of the preceding methods, wherein the donor organ is from a different species as the recipient
1.35. Method I or any of the preceding methods, wherein the T3 and VIP nanoparticles are administered to an organ up to 24 hours before transplantation.
1.36. Method of method 1.35 where the T3 and VIP nanoparticles are administered from about 1, 2, 3, 4, 6, 8, 10, 12, 14, 16, 18, 20, 22, and 24 hours prior to transplantation.
1.37. Method I or any of the preceding methods, wherein the T3 and VIP are delivered in combination nanoparticles which comprise both T3 and VIP.
1.38. Method I or any of the preceding methods wherein the T3 nanoparticles and VIP nanoparticles are administered to a donor organ in a composition which comprises T3 and/or VIP nanoparticles.
1.39. Method I or any of the preceding methods wherein the T3 nanoparticles and VIP nanoparticles are administered to a donor organ in a formulation which comprises T3 and/or VIP nanoparticles.
1.40. Method I or any of the preceding methods wherein the donor organ is a human donor organ.
1.41. Method I or any of the preceding methods wherein an effective amount of T3 nanoparticles and VIP nanoparticles are administered simultaneously to a donor organ.
1.42. Method I or any of Methods 1.1-1.40 wherein an effective amount of T3 nanoparticles are first administered to a donor organ followed by a second administration of an effective amount of a VIP nanoparticle formulation to said donor organ.
1.43. Method I or any of Methods 1.1-1.40 wherein an effective amount of VIP nanoparticles are first administered to a donor organ followed by a second administration of an effective amount of a T3 nanoparticle formulation to said donor organ.
1.44. Method I or any of the preceding methods, wherein an effective amount of a formulation comprising T3 nanoparticles, VIP nanoparticles, and dopamine are administered to a donor organ (e.g., either prior, during, or after transplantation) in order to reduce the potential for rejection (e.g., acute or chronic rejection).
1.45. Method of 1.44, wherein the formulation further comprises an anti-rejection agent.
1.46. Method I or any of the preceeding methods, wherein an effective amount of a T3, VIP, or T3/VIP nanoparticle formulation is administered to a donor organ (e.g., prior, during, or after transplantation) in order to reduce the risk or occurrence of acute or chronic rejection resulting from reperfusion injury.
1.47. Method I or any of the preceeding methods, wherein an effective amount of a T3, VIP, or T3/VIP nanoparticle formulation is administered to a donor organ (e.g., prior, during, or after transplantation) and wherein the formulation prevents or limits reperfusion injury.
1.48. Method of 1.47, wherein the prevention or limitation of reperfusion injury reduces the risk of either acute or chronic transplant rejection, wherein the reduction of the risk is relative to an organ that was not treated or administered a T3, VIP, T3/VIP nanoparticle, and wherein the limitation of reperfusion injury is relative to the damage to an untreated organ.
1.49. Method I, or any of the preceding methods, wherein the nanoparticle formulation is administered to a patient in need thereof after the donor organ has been transplanted, and wherein the amount is effective to prevent or reduce the risk of organ rejection (e.g., acute or chronic rejection).
1.50. Method of any of the preceding methods, wherein an effective amount of a T3, VIP, T3/VIP nanoparticle formulation is administered to a donor organ in an amount which is effective to attenuate acute rejection, or limit recurrent rejection episodes, and wherein this attenuation or limitation of recurrent rejection is increased relative to an organ that is not treated with a T3, VIP, T3/VIP nanoparticle formulation.

The present invention provides that an effective amount of a T3, VIP, or T3/VIP nanoparticle formulation may be administered to donor organs that have been transplanted from a donor to a recipient, or intended for transplant from a donor to recipient. “Nanoparticle” as used herein, refers to a nanoparticle wherein T3 and/or VIP are encapsulated or immobilized by a bioabsorbable polymer having any of the following characteristics:
a. Wherein the polymer comprises chitosan.
b. Wherein the polymer comprises poly(lactic-co-glycolic acid) (PLGA) or polylactic acid (PLA), e.g., PLGA having 50/50 co-polymerization of D,L-lactic acid and glycolic acid.
c. Wherein the polymer comprises chitosan crosslinked using glutaraldehyde.
d. Wherein the polymer comprises chitosan linked to bile acids.
e. Wherein the polymer comprises chitosan linked to PLGA, e.g., using glutaraldehyde as crosslinker.
f. Any of the foregoing wherein the nanoparticle comprises acetic acid and/or an acetic acid analogue, e.g., Dicholoroacetic acid (“DCA”).
g. Any of the foregoing wherein the nanoparticles have an average diameter of 50-1000 nm, e.g., 100-500 nm or 50-250 nm.
h. Any of the foregoing wherein the nanoparticles have a zeta potential of 10-100 mV.
i. Any of the foregoing wherein the nanoparticle comprises a second pharmacologically active ingredient.

It is contemplated that the T3 nanoparticles and VIP nanoparticles of Method I and methods 1.1-1.50, may have any of the nanoparticle characteristics that are disclosed herein (e.g., in the foregoing list a.-i.)

It is also contemplated that the T3 nanoparticles and VIP nanoparticles of Method I and methods 1.1-1.50, may have any of those characteristics disclosed in paragraph [00027], items a-i.

It is contemplated that the T3 nanoparticles and VIP nanoparticles of any of the embodiments disclosed herein may have any of the nanoparticle characteristics disclosed herein.

It is contemplated the T3 nanoparticles and VIP nanoparticles of any of the embodiments disclosed herein may have any of the characteristics disclosed in paragraph [00027], items a-i.

It is contemplated that the relevant T3 nanoparticles and VIP nanoparticles of any of the embodiments disclosed herein may be utilized in any of Method I and/or methods 1.1-1.50.

In one further aspect, the invention provides T3 and/or VIP nanoparticles, wherein the T3 and/or VIP is encapsulated or immobilized by a bioabsorbable polymer (e.g. having any of the characteristics of foregoing list a.)-i.)), wherein the bioabsorbable polymer is chitosan for example, wherein the chitosan has any of the following characteristics:
j.) The chitosan is derived from fungus, e.g. mushroom or mold;
k.) The chitosan is derived from fungus, wherein the fungus is an Aspergillus, e.g., Aspergillus niger;
l.) The chitosan is derived from mushroom, wherein the mushroom is an Agaricus, e.g., Agaricus bisporus;
m.) Any of the foregoing wherein the chitosan has a molecular weight range of about Mv 30,000-220,000;
n.) Any of the foregoing wherein the chitosan a molecular weight range of about Mv 30,000-60,000;
o.) Any of the foregoing wherein the chitosan has a range of apparent viscosity (e.g. at 1% solution in 1% acetic acid) of about <20 mPa·s to 90 (+/−30 mPa·s); e.g. <20 mPa·s, 40 (+/−20 mPa·s), 55 (+/−25 mPa·s), 90 (+/−30 mPa·s);
p.) Any of the foregoing wherein the chitosan has a degree of acetylation (% mol) in a range of about 10%-40%;
q.) Any of the foregoing wherein the chitosan has a degree of acetylation (% mol) in a range of about e.g, 10%-20%, 15%-25%, 20%-30%, or 30%-40%.
r.) Any of the foregoing wherein the T3 is encapsulated within the chitosan, and wherein there is a greater ratio of chitosan present in the nanoparticle relative to the amount of PLGA, e.g. a relative ratio amount of 80/20, chitosan to PLGA, (e.g., % w/w 80/20, chitosan to PLGA)

The above measurements may be carried out by any means known in the art. For example, it is contemplated that the viscosity of chitosan solutions may be measured at room temperature using a Brookfield type digital viscometer, e.g., DV-11+Pro. In another example, it is contemplated that the viscosity may be measured using a Ubbelohde type viscometer. In such an example, it is contemplated that the viscometer could be connected to a visco-clock to record the time of the passing solution.

In one aspect, the present invention provides for T3, VIP, or T3/VIP nanoparticles wherein the nanoparticle comprises chitosan, e.g., having any of the characteristics of foregoing list a.)-j.), and PLGA, wherein the relative ratio of chitosan to PLGA may be altered to adjust the release of the active ingredient, e.g. T3. Without being bound by theory, it is believed that chitosan is hydrophilic. Therefore, where the active ingredient may possibly be hydrophobic (e.g. T3) the addition of more chitosan relative to PLGA may result in a nanoparticle wherein the active ingredient is quickly released upon application or administration, e.g., a relative ratio amount of 80/20, (e.g., % w/w 80/20, chitosan to PLGA) chitosan to PLGA, or a relative ratio amount of 90/10 (e.g., % w/w 90/10, chitosan to PLGA) chitosan to PLGA. Without being bound by theory, where the active ingredient is more hydrophobic, the addition of more PLGA, relative to the amount of chitosan, may result in a nanoparticle wherein the active ingredient is more slowly released, e.g., a relative ratio of 20/80 chitosan to PLGA (e.g., % w/w 20/80, chitosan to PLGA), or 10/90 chitosan to PLGA (e.g., % w/w 10/90, chitosan to PLGA)

“T3 nanoparticles” as discussed herein, can refer to nanoparticles wherein T3 is encapsulated or immobilized by a bioabsorbable polymer. It is contemplated that the bioabsorbable polymer may have any of the characteristics that are disclosed herein. “VIP nanoparticles” as discussed herein, can refer to nanoparticles wherein VIP is encapsulated or immobilized by a bioabsorbable polymer. It is contemplated that the bioabsorbable polymer may have any of the characteristics that are disclosed herein.

It is contemplated that the T3 nanoparticles and/or VIP nanoparticles discussed and disclosed herein may be utilized in any of the appropriate embodiments and methods disclosed herein, e.g. Method I and/or methods 1.1-1.50.

It is contemplated that the T3 nanoparticles and VIP nanoparticles discussed in the embodiments and methods disclosed throughout the specification and claims can have any of the nanoparticle characteristics disclosed throughout the specification and claims, e.g. the characteristics noted in paragraph [00027], items a-i.

In one embodiment, the nanoparticle comprises T3 and VIP, and a bioabsorbable polymer, wherein the bioabsorbable polymer immobilizes T3 to the outside of the nanoparticle and encapsulates VIP on the inside of the nanoparticle.

In one embodiment, the T3 is covalently linked to the bioabsorbable polymer, for example via the hydroxy on the phenyl moiety, and VIP is encapsulated or immobilized in the bioabsorbable polymer. Such compositions can be formed using activated T3 which is activated at the phenolic hydroxy with a suitable linker and protected at the amino moiety. The amino-protected T3 is then linked to the nanoparticle, for example via the phenolic hydroxy, e.g. by using an activated linker group, for example a moiety capable of coupling to an amine group on the bioabsorbable polymer, for example the amino moieties on chitosan.

In one embodiment, the invention provides a nanoparticle comprising T3 and/or VIP, wherein the T3 is an activated T3 which is substituted on the phenolic hydroxy group with an epoxide moiety of formula [CH2-O—CH]—[CH2]ₙ— and which is amino protected. For example, the invention provides a T3/VIP nanoparticle compound wherein T3 is formula 1:

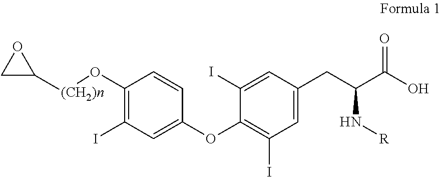

wherein n is an integer selected from 1 through 5, and R is an amino protecting group, e.g., butoxycarbonyl (BOC).

In one embodiment, the T3 and/or VIP nanoparticle can comprise a T3 which may be activated, for example using an epoxyalkyl of formula [CH2-O—CH]—[CH2]ₙ—X wherein n=1-5 and X is halogen, e.g. bromine, e.g. according to a synthesis as shown in FIG. 10. The resulting compound is then, if necessary, selectively deprotected to release the carboxy moiety, for example,

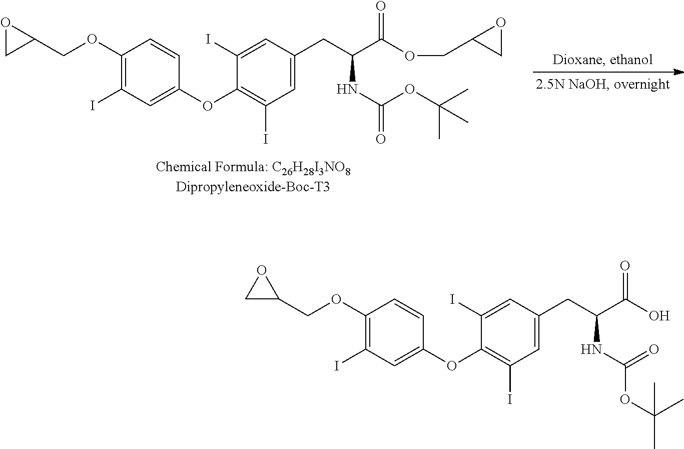 [SMILES: CC(C)(C)OC(=O)N[C@@H](Cc1cc(I)c(Oc2ccc(OCC3CO3)c(I)c2)c(I)c1)C(=O)OCC1CO1 ; CC(C)(C)OC(=O)N[C@@H](Cc1cc(I)c(Oc2ccc(OCC3CO3)c(I)c2)c(I)c1)C(=O)O]

to provide T3 which is activated at the phenolic hydroxy (here, with propylene oxide) and amino-protected (here, with BOC).

The activated T3 may be attached to the bioabsorbable polymer, for example, T3 having an epoxy linker moiety and an amino-protecting group is reacted with a bioabsorbable polymer having amino groups, then deprotected to provide a nanoparticle covalently linked to T3. This reaction may be carried out in the presence of a stabilizer, such as polyvinyl alcohol, e.g. PVA 1% w/v, in an appropriate solvent, for example dimethylsulfoxide, e.g. DMSO (0.1% v/v) and acetic acid (0.1% v/v), which solvents are removed afterwards by dialysis. The number of T3 moieties attached to the nanoparticle may vary based on the reaction conditions and amount of reactant used, but if these conditions are kept constant, the distribution of variation will be low. Typically, the nanoparticle will comprise 20-200 T3 moieties, e.g., about 50 per nanoparticle. The amount of T3 in a batch can be assayed, e.g., as described below, by separating the nanoparticles by filtration or centrufugation, weighing, degrading the T3/VIP nanoparticle in strong base, and measuring by HPLC.

In one example, the T3 nanoparticles and/or VIP nanoparticles comprise T3 and/or VIP, and the following components:

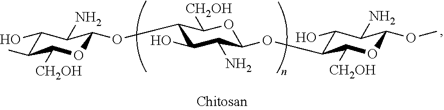

In one embodiment, the invention provides a method for treating an acute cardiac condition, e.g. cardiac arrest, cardiac arrhymia, or cardiac insufficiency, comprising administering an effective amount of a T3/VIP-nanoparticle to a patient in need thereof, wherein the T3/VIP-nanoparticle comprises a bioabsorbable polymer, for example as described above.

In one embodiment, the present invention provides that the T3 can refer to a T3 analog, and/or a T3-like peptide, and/or a functional variant.

In one embodiment the present invention provides that the VIP can refer to a VIP analog, and/or a VIP-like peptide, and/or a functional variant.

Nanoparticle production is generally described in the Applicant's own publications: US 20110142947 A1, and WO 2011/159899, the contents of each of which are incorporated herein by reference in their entireties. The T3 nanoparticles and VIP nanoparticles discussed in the Applicant's aforementioned publications may be utilized in any of the relevant embodiments or methods discussed and disclosed herein, e.g., Method I and/or methods 1.1-1.50.

The examples provided herein are merely examples which should not be used to limit the scope of the claim construction or interpretation.

Alternative combinations and variations of the examples provided will become apparent based on this disclosure. It is not possible to provide specific examples for all of the many possible combinations and variations of the embodiments described, but such combinations and variations may be claims that eventually issue.

1. A method for preventing or reducing the risk of organ rejection of a donor organ, comprising administering or treating a donor organ with an effective amount of a formulation comprising T3, VIP, and/or T3/VIP nanoparticles.

2. The method of claim 1, wherein the organ is intended for transplant from donor to a recipient.

3. The method of claim 1, wherein the T3 and/or VIP nanoparticles are administered prior to transplantation of an organ.

4. The method of claim 1, wherein the T3 and/or VIP nanoparticles are administered contemporaneously with the transplantation of an organ.

5. The method of claims 1, wherein the T3 and/or VIP nanoparticles are administered both prior to, and contemporaneously, with the transplantation of an organ.

6. The method of claim 1, wherein an effective amount of a combination T3/VIP nanoparticle are administered.

7. The method of claim 1, wherein an effective amount of T3 and/or VIP nanoparticles are administered in combination with an additional thyroid hormone.

8. The method of claim 7 wherein the additional thyroid hormone is thyroxine (T4).

9. The method of claim 1, wherein the donor organ is selected from the group consisting of: kidney, heart, lung, combined heart/lung, liver, pancreas, islet cell, face, hand, uterus, bone marrow, eye, cornea, trachea, bowel, e.g. small bowel, skin, muscles or limb, oesophagus, and nervous tissue.

10. The method of claim 9, wherein the organ is heart.

11. The method of claim 1, comprising the administration of an effective amount of a formulation comprising T3 nanoparticles, VIP nanoparticles, and dopamine to a donor organ, and wherein the formulation is effective to reduce the risk of organ rejection.

12. The method of claim 11, wherein the formulation further comprises an anti-rejection agent.

13. A method of preventing or reducing the risk of organ transplant rejection in a donor organ, wherein the method comprises the administration of an effective amount of a T3, VIP, or T3/VIP nanoparticle formulation to a donor organ, and wherein the amount of the nanoparticle formulation is effective to prevent or reduce the risk of acute or chronic rejection resulting from reperfusion injury.

14. Method of claim 13, wherein an effective amount of a T3, VIP, or T3/VIP nanoparticle formulation is administered to a donor organ and wherein the formulation prevents reperfusion injury.

15. Method of claim 13, wherein the prevention of reperfusion injury reduces the risk of either acute or chronic transplant rejection, wherein the reduction of the risk is relative to an organ that was not treated or administered a T3, VIP, T3/VIP nanoparticle.

16. A method of preventing or limiting reperfusion injury in a donor organ comprising administering or treating said organ with an effective amount of a formulation comprising T3, VIP, and/or T3/VIP nanoparticles.
